SEQUENCE LISTING (17 sequences)

SEQ ID NO: 1 - DNA, 293 bp, Artificial Sequence
  primer
ggccacgcgtcgactagtacgggggggggggggggggggagcataaacaaataaataaagacaactataaaagaaaaaaaaggaataaattaaaaaaatggtaaagctcacacaagttgcagcaatactcttaatcggggccttcttcttgatagcctccccttccatagctacacaaacatctcatcccgcaaaattccacgttgagggagaggtatactgcaatgtttgtcacagcagaaatttaatcaatgaactcagcgaacgcatggcaggggcacaagtgcaattgg

SEQ ID NO: 2 - DNA, 288 bp, Artificial Sequence
  primer
ggccacgcgtcgactagtacgggggggggggggggggataaacaaatgaataaagacaactaaaaaagaaaaaaaaggaataaattaaaaaaatggtaaagctcacacaagttgcagcaatactcttaatcggggccttcttcttgatagcctccccttccatagctacacaaacatctcatcccgcaaaattccacgttgagggagaggtatactgcaatgtttgtcacagcagaaatttaatcaatgaactcagcgaacgcatggcaggggcacaagtgcaattgg

SEQ ID NO: 3 - DNA, 263 bp, Artificial Sequence
  primer
ggccacgcgtcgactagtacggggggggggggggaaaaaaaagaaaaaaagaaaaaaagaaaaaaatatggtaaagctcacacaagttgcagcaatactcttaatcggggccttcttcttgatagcctccacttccatagctacacaaacatctcatcccgcaaaattccacgttgagggagaggtatactgcaatgtttgtcacagcagaaatttaatcaatgaacttagcgaacgcatggcaggggcacaagtgcaattgg

SEQ ID NO: 4 - protein, 25 aa, Plantago lanceolata
MVKLTQVAAILLIGAFFLIASPSIA

SEQ ID NO: 5 - DNA, 704 bp, Plantago lanceolata
acacaaacatctcatcccgcaaaattccacgttgagggagaggtatactgcaatgtttgtcacagcagaaatttaatcaatgaactcagcgaacgcatggcaggggcacaagtgcaattggattgcaaagatgattctaaaaaagtcatatactctatagggggtgagactgatcaagatggtgtttaccgcctgcctgttgtaggctatcacgaagattgtgaaatcaaactagtgaagagcagcaggcccgattgtagtgaaattccgaaacttgcaaagggaacaattcaaacctcgaaagtggacctttcaaaaaacacaaccatcaccgaaaaaacacgtcatgtcaagccactgagctttcgcgcaaagacggatgcccctgggtgttaaaggatgctgcagagggcgattaattgtacaagtccaattttcataataacaagcgttcaatgtgattcctttttcttgttttcttgttttcttttttgttcccccagttttgtagtagtattcaaagttcaataaggtgtttccagaccctggttgaggttggtttctcaggatactgatgaacctttttattatctatgagtagttatacgcaaaagtttggatagtgtttatatttaaatggaatttgatgttcttacaattcgaaaaaaaaaaaaaaaaaaaaaaaaaaaaaaaaaaaaaaaaaa

SEQ ID NO: 6 - DNA, 692 bp, Plantago lanceolata
acacaaacatctcatcccgcaaaattccacgttgagggagaggtatactgcaatgtttgtcacagcagaaatttaatcaatgaacttagcgaacgcatggcaggggcacaagtgcaattggattgcaaagatgattctaaaaaagtcatatactctatagggggtgagactggtcaagatggtgtttaccgcctgcctgttgtaggctatcacgaagattgtgaaatcaaactagtgaagagcagcaggcccgattgtagtgaaattccgaaacttgcaaagggaacaattcaaacctcgaaagtggacctttcaaaaaacacaaccatcaccgaaaaaacacgtcatgtcaagccactgagctttcgcgcaaagacggatgcccctgggtgttaaaggatgctgcagagggcgattaattgtacaagtccaattttcttaataacaagcgttcaatgtgattcctttttcttgttttcttgttttcttttttgttcccccagttttgtagtagtattcaaagttcaataaggtgtttccagaccctggttgaggttggtttctcaggatactgatgaacctttttattatctatgagtagttatacgcaaaagttttgcaagcagtgagaaatcactgtagtttatatttaaatggaatttgatgttcttacaaaaaaaaaaaaaaaaaaa

SEQ ID NO: 7 - DNA, 679 bp, Plantago lanceolata
acacaaacatctcatcccgcaaaattccacgttgagggagaggtatactgcaatgtttgtcacagcagaaatttaatcaatgaacttagcgaacgcatggcaggggcacaagtgcaattggattgcaaagatgattctaaaaaagtcatatactctatagggggtgagactggtcaagatggtgtttaccgcctgcctgttgtaggctatcacgaagattgtgaaatcaaactagtgaagagcggcaggcccgattgtagtgaaattccgaaacttgcaaagggaacaattcaaacatcgaaagtggacctttcaaaaaacacaaccatcaccgaaaaaacacgtcatgtcaagccactgagctttcgcgcaaagacggatgcccctgggtgttaaaggatgctgcagagggcgattaattgtacaagtccaattttcttaataacaagcgttcaatgtgattcctttttcttgttttcttgttttcttttttgttcccccagttttgtagaagtattcaaagttcaataaggtgtttccagaccctggttgaggttggtttctcaggatactgatgaacctttttattatctatgagtagttatacgcaaaagtttggatagtgtttatatttaaatggaatttgatgttcttacaaaaaaaaaaaaaaaaaaaaaaa

SEQ ID NO: 8 - protein, 131 aa, Plantago lanceolata
TQTSHPAKFHVEGEVYCNVCHSRNLINELSERMAGAQVQLDCKDDSKKVIYSIGGETDQDGVYRLPVVGYHEDCEIKLVKSSRPDCSEIPKLAKGTIQTSKVDLSKNTTITEKTRHVKPLSFRAKTDAPGC

SEQ ID NO: 9 - DNA, 37 bp, Artificial Sequence
  primer
ggccacgcgtcgactagtacttttttttttttttttt

SEQ ID NO: 10 - DNA, 32 bp, Artificial Sequence
  primer
cuacuacuacuaggccacgcgtcgactagtac

SEQ ID NO: 11 - DNA, 20 bp, Artificial Sequence
  primer
cayccngcnaarttycaygt

SEQ ID NO: 12 - DNA, 29 bp, Artificial Sequence
  primer
cggaatttcactacaatcgggcctgccgc

SEQ ID NO: 13 - DNA, 33 bp, Artificial Sequence
  primer
ccaattgcacttgtgcccctgccatgcgttcgc

SEQ ID NO: 14 - DNA, 20 bp, Artificial Sequence
  primer
acacaaacatctcatcccgc

SEQ ID NO: 15 - DNA, 35 bp, Artificial Sequence
  primer
ctcgagaaaagagagacacaaacatctcatcccgc

SEQ ID NO: 16 - DNA, 23 bp, Artificial Sequence
  primer
gggaattcttaacacccaggggc

SEQ ID NO: 17 - DNA, 36 bp, Artificial Sequence
  primer
ggccacgcgtcgactagtacgggnngggnngggnng

(19) United States
(12) Patent Application Publication
(10) Pub. No.: US 2004/0071717 A1
(43) Pub. Date: Apr. 15, 2004

(54) Recombinant DNA encoding the major allergen of plantago lanceolata pollen, Pla I 1, and applications thereof

(75) Inventors: Belen Calobozo Freile, Madrid (ES); Araceli Diaz Perales, Madrid (ES); Domingo Barber Hernandez, Madrid (ES); Gabriel Salcedo Duran, Madrid (ES); Florentino Polo Corrales, Madrid (ES)

Correspondence Address: DARBY & DARBY P.C., P. O. BOX 5257, NEW YORK, NY, 10150-5257, US

(73) Assignee: ALK-ABELLO A/S

(21) Appl. No.: 10/161,551

(22) Filed: Sep. 3, 2002

Related U.S. Application Data
(60) Provisional application No. 60/294,672, filed on May 30, 2001.

(30) Foreign Application Priority Data
May 30, 2001 (DK) PA 2001 00857

(51) Int. Cl. A61K 39/00; C07H 21/04; C07K 14/415

(52) U.S. Cl. 424/185.1; 530/370; 536/23.6

(57) ABSTRACT

A nucleic acid molecule encoding a peptide or protein comprising at least one epitope of the major allergen of Plantago lanceolata, Pla I 1, wherein the nucleic acid molecule a) has the sequence of SEQ ID NOS.: 5-7, b) is a fragment of the sequence SEQ ID NOS.: 5-7, c) has a sequence encoding the amino acid sequence of SEQ ID NO.: 8 or a fragment thereof, d) has a sequence hybridising to SEQ ID NOS.: 5-7 under stringent conditions, e) has a sequence derivable by degeneration of SEQ ID NOS.: 5-7, or f) a complementary strand of any of the sequences a)-e).

This application claims the priority of U.S. Provisional Application No. 60/294,672, filed on May 30, 2001 which is hereby incorporated hereby by reference in its entirety.

### FIELD OF THE INVENTION

The present invention relates to a nucleic acid molecule comprising at least one epitope of the major allergen of Plantago lanceolata, Pla I 1.

### BACKGROUND OF THE INVENTION

Type I allergies affect millions of people worldwide, and its incidence has increased over the last few years in developed countries, leading to rising human and economic costs (1). Pollen allergens are proteins or glycoproteins capable of eliciting IgE-mediated allergic diseases, such as hay fever and 2 o asthma, in approximately 17% of the population who are genetically predisposed to develop allergies.

The current treatment for these diseases consists primarily in symptomatic relief. Patients are treated with drugs, such as anti-histamines and steroids, which do not suppress the formation of IgE antibodies and often have harmful side effects. As stated in the WHO Position Paper (2), immunotherapy is the only treatment that may affect the natural course of allergic diseases, and it also may prevent the development of asthma in patients with allergic rhinitis (2). Immunotherapy modulates the immune response in patients throughout the administration of increasing amounts of the appropriate allergenic extract. However, allergenic extracts used therapeutically are crude mixtures of proteins and non-protein components isolated from natural sources that comprise a number of constituents bearing no relation to the allergen or few allergens which are responsible for patient's hypersensitivity. Although in the last years, with the advent of hybridoma technology, significant progresses have been made in the standardization of allergenic extracts through the determination of major allergen content by monoclonal antibody-based immunoassays (3), all the patients sensitive to a given allergenic extract proceed receiving the same complex mixture containing all the constituents of the extract. This may give rise to the onset of side reactions arising from additional IgE antibodies towards all the protein components of the extract including those allergens different from the major allergen Pla I 1.

As far as allergy diagnostics is concerned, the use of whole allergenic extracts for cutaneous tests precludes the identification of the specific allergens responsible for patient's sensitization.

The approach adopted by many researchers to circumvent all these drawbacks has been to use biochemical separation and purification techniques to isolate the individual allergens. Nevertheless this approach, that may be very useful for the characterization of the allergens, is not adequate for the preparation of allergens on an industrial scale as the processes are extremely labour intensive and the yield of allergen purified from usually expensive natural sources is very low. For these reasons, great attention has been paid to the recombinant DNA technologies for the synthesis of allergenic proteins. Recombinant allergens can be obtained on a large scale by using microbial expression systems that may be grown on large fermenters. Thus, these techniques allow the production of recombinant allergens in a consistently pure state with a better yield. Besides, using the rDNA approach it is possible to express epitopic fragments or modified allergens for convenient use in diagnosis or treatment of allergic diseases. An increasing number of researchers are nowadays using the rDNA technology for the study of allergens, and, for instance, some allergens from mites, grasses, trees, moulds, etc, have recently been cloned and the respective recombinant allergens expressed (4).

The pollens of plants belonging to genus Plantago constitute a major source of aeroallergens and may account for a relevant proportion of the pollinosis in a number of countries worldwide. The genus Plantago belongs to the family Plantaginaceae and comprises about 250 species. One of the most common species is Plantago lanceolata (English plantain or ribwort), that is distributed in the temperate zones of Europe, Australia and North America (5-8). P. lanceolata pollen has been associated with hay fever since the beginning of this century (9-11), and this weed has been considered as one of the most important dicotyledons that cause allergic diseases (12).

In the past years, several studies in different countries have shown the clinical importance of this species. The highest incidence of allergy to Plantago lanceolata has been described in Australia (13) and in the Mediterranean area (14). For instance, Bousquet et al. (15) examined patients with pollinosis and found that 36% of them were sensitized to English plantain in Montpellier (France). In England, two different studies showed that more than 20% of patients with seasonal respiratory allergy gave positive skin reactions to plantain (16, 17). Likewise, a high prevalence of allergy to this pollen has been reported in different cities of Spain (18-20). Nevertheless, the true role of P. lanceolata pollen in the aetiology of pollinosis is unclear, because sensitization to plantain alone is unusual. Patients sensitized to plantain are usually also sensitive to others plants that pollinate in the same season, especially grass pollens (17-21).

Despite the above-mentioned publications, few data on the characterization of the major plantain allergens have been reported, and only some studies dealing with the identification of P. lanceolata allergens have been published so far (17, 21, 22).

WO 98/59051 discloses cloning of Ole e 1 allergens from olive. It is known that this sequence shares homology with an 8 amino acid sequence from Pla I 1.

A sequence from birch (Betula verrucosa) is available in databases under the accession number Y14038. This sequence encodes a protein that shares a high degree of homology with Ole e 1 allergens.

The major allergen of P. lanceolata pollen has recently been identified, purified and partially characterized in terms of both physicochemical and immunochemical properties (23). Pla I 1 is a microheterogeneous glycoprotein with an apparent molecular weight in the range of 16 to 20 kDa. Sixteen amino acid residues from the N-terminal end were determined. Prevalence of specific IgE to pure Pla I 1 in plantain-sensitized patients was 86%, and it contributes about 80% of the total IgE-binding capacity of the plantain pollen extract. These data demonstrated that Pla I 1 is the most clinically relevant allergen from P. lanceolata pollen.

### SUMMARY OF THE INVENTION

The object of the present invention is to provide the nucleotide and amino acid sequence of Pla I 1.

This object is obtained with the present invention, which relates to a nucleic acid molecule encoding a peptide or protein comprising at least one epitope of the major allergen of Plantago lanceolata, Pla I 1, wherein the nucleic acid molecule a) has the sequence selected from the group consisting of SEQ ID NOS.: 5-7, b) is a fragment of the sequence selected from the group consisting of SEQ ID NOS.: 5-7, c) has a sequence encoding the amino acid sequence of SEQ ID NO.: 8 or a fragment thereof, d) has a sequence hybridising to the sequence set forth in any one of SEQ ID NOS.: 5-7 under stringent conditions, e) has a sequence derivable by degeneration of the sequence set forth in any one of SEQ ID NOS.: 5-7, or f) a complementary strand of any of the sequences a)-e).

Molecular cloning techniques were used to isolate cDNA clones encoding Pla I 1 allergen, and to determine the nucleotide sequence of the clones. The amino acid sequence is deduced from the nucleotide sequence to obtain the complete chemical structure of the protein. The process involves isolating RNA from P. lanceolata pollen and synthesizing the cDNA using reverse transcriptase. cDNA coding for Pla I I is specifically amplified by PCR using specific primers derived from the N-terminal sequence of the natural allergen. 5′-extension of Pla I 1-encoding cDNA using internal specific primers allows the determination of the nucleotide sequence coding for the signal peptide and the N-terminal end of Pla I 1. Finally, Pla I 1-cDNA is amplified by PCR using primers specific for both the N- and C-terminal ends of the mature protein, and full-length clones coding for Pla I 1 are inserted into a vector and the recombinant allergen expressed in a transformed host.

Pollen grains consist of a rigid exterior wall enclosing a number of cells, and the protein allergens are present intracellularly. Thus, in order to isolate mRNA from pollen the rigid exterior wall has to be disrupted, the cells should be lysed and the mRNA extracted from the resulting mixture. The isolation is particularly difficult due to the volatile nature of mRNA, which typically only exists for a few minutes in living cells.

Prior to this invention attempts have been made to isolate mRNA for Pla I 1 from P. lanceolata pollen with no success. In particular, a number of different methods of grain wall disruption, cell lysis and mRNA extraction was used without success. In accordance with the present invention isolation of mRNA for Pla I 1 was achieved using a selected combination of methods. Specifically, the grain wall was disrupted using a modified sonification procedure, wherein the period of treatment was prolonged strongly as compared to prior art sonification procedures. This modified sonification was used in combination with a lysis buffer comprising guanidinium thiocyanate and in combination with an extraction agent based on phenol-chloroform.

The present invention provides isolated nucleic acid molecules coding for epitopes of the major allergen of P. lanceolata pollen, which represents an improvement in diagnosis and treatment of allergy diseases by providing the means for overcoming the lack of pure allergens or peptides corresponding to allergenic portions thereof, as referred to above. This allergen, though constituting the major allergenic component of Plantago plant pollen, remained molecular uncloned prior to this invention.

The material and information obtained allow the modification of the nucleic acid molecules and hence the alteration of specific amino acid residues in the protein in order to identify specific IgE-binding epitopes. The identification of IgE-binding epitopes allows the manufacture of modified recombinant allergens with diminished IgE-binding capability. Modified recombinant allergens with diminished IgE-binding capability may be used for effective treatment of allergic patients, as larger doses can be administered with lower risk of adverse side-reactions, such as anaphylactic reactions. In the same way, it is also possible to design short peptides derived from the Pla I 1 sequence that could be potentially used as a vaccine by regulating T-cell responses that control IgE antibody production in allergic patients. Also, the recombinant allergen can be chemically modified. An additional aspect of the present invention is the use of the recombinant allergen for diagnosing allergic reactions to pollens from P. lanceolata and cross-reactive species. The diagnostic methods are based on antigen-antibody reactions and can then be designed for both in vivo and in vitro tests.

The present invention further relates to the following:

A recombinant protein or peptide comprising at least one epitope of the major allergen of Plantago lanceolata, Pla I 1 having the amino acid sequence corresponding to a nucleic acid sequence according to the present invention disclaiming the amino acid sequence consisting of amino acids 1-16 of SEQ ID NO.: 8 and fragments thereof.

The protein or peptide according to the present invention for use as a pharmaceutical.

Use of the protein according to the present invention for the manufacture of a pharmaceutical for preventing, alleviating or treating allergic reactions in a subject.

An expression vector adapted for transformation of a host, the vector comprising a nucleic acid molecule according to the present invention.

A host cell comprising the expression vector according to the present invention.

A method of producing a recombinant peptide or protein comprising at least one epitope of the major allergen of Plantago lanceolata, Pla I 1, the method comprising culturing a host cell according to the present invention under conditions such that said Pla I 1 nucleotide sequence is expressed and said peptide or protein is produced, and isolating said peptide or protein.

A pharmaceutical composition comprising as an active substance, a recombinant peptide, or protein according the present invention.

A method of preventing, alleviating or treating allergic reactions in a subject comprising administering to the subject a recombinant peptide or protein according to the present invention, or the pharmaceutical composition the present invention.

An in vitro method of diagnosing or prognosticating allergy to Pla I 1 allergen in a subject comprising collecting a sample from the subject and determining the level of IgE antibodies to the protein or peptide according to the present invention.

An in vivo method of diagnosing or prognosticating allergy to Pla I 1 allergen in a subject comprising subjecting a subject to the protein or peptide according to the present invention and monitoring the reaction of the subject.

A reagent for use in in vitro or in vivo diagnosing or prognosticating allergy to Pla I 1 allergen in a subject, wherein the reagent contains the protein or peptide according to the present invention.

A method of predicting the effect of allergy vaccination comprising using the protein or peptide according to the present invention.

### DESCRIPTION OF SEQUENCES

SEQ ID NOS.: 1-3. Nucleotide sequence of three cDNA clones obtained using the 5′RACE system. Asterisks (*) indicate sequence identity in the three sequences. The translation start codon is underlined, the leader peptide sequence is in italics, and the nucleotide sequence of the oligonucleotide encoding for the N-terminal end of the mature protein (primer Pla4, Table 1) is in bold type. A nucleotide sequence complementary to that of primer Pla 3 used in 5′RACE can be observed (discontinuous underlining) at the end of the sequence of each clone.

SEQ ID NO.: 4. Amino acid sequence of the leader peptide of Pla I 1 derived from the nucleotide sequence of three cDNA clones. Dashes indicate identity with the amino acid residue in the upper line.

SEQ ID NOS.: 5-7. Nucleotide sequence of cDNA clones encoding Pla I 1 starting from the N-terminal end of the mature protein. Asterisks (*) indicate sequence identity among the clones. The sequence corresponding to the mature protein is in capital letters, and the 3′ untranslated region is in lower case. The stop codon is in bold type. The name of each clone according to IUIS nomenclature rules is indicated in bold type at the end of the sequence.

SEQ ID NO.: 8. Translated amino acid sequence from nucleotide sequence of Pla I 1-cDNA clones. Dashes indicate identity with the amino acid residue in the upper line. The potential N-glycosylation site is in a box. The name of each clone according to IUIS nomenclature rules is indicated in bold type at the end of the sequence.

### BRIEF DESCRIPTIONS OF DRAWINGS

FIG. 1. SDS-PAGE. Analysis of purified nPla I 1 (lane 1) and rPla I 1.0101 (lane b). Lane c: rPla I 1.0101 after treatment with PNGase F. Eight μg of protein was loaded per lane. Staining was carried out with Coomassie Brilliant Blue.

FIG. 2. Circular dichroism spectra of natural and recombinant Pla I 1 in the far UV. Values are expressed as mean residue ellipticities (0 mrw), on the basis of 113 as the mean residue weight in Pla I 1.

FIG. 3. Inhibition of specific IgE-binding to nPla I 1. 96-well ELISA plates were coated with pure natural Pla I 1, and the binding of specific IgE from a pool of sera from plantain-allergic patients was inhibited by the addition of rPla I 1.0101. An inhibition curve with the natural allergen was used for comparison. Detection of bound IgE was accomplished with horseradish peroxidase-labeled anti-human IgE rabbit antibodies and orto-phenylendiamine (OPD). Absorbance was measured at 490 nm with a reference filter at 650 nm.

### DETAILED DESCRIPTION OF THE INVENTION

The nucleotide sequences coding for three isoallergenic variants of the major allergen of Plantago lanceolata pollen (English plantain), Pla I 1.0101, Pla I 1.0102 and Pla I 1.0103 are set forth in SEQ ID NOS.: 5-7, and the amino acid sequences thereof, are set forth in SEQ ID NO.: 8. The isoallergenic variant Pla I 1.0101 has been expressed, purified and characterized. The nucleotide sequences of Pla I 1 variants encode a 131 residue mature processed protein. Recombinant Pla I 1.0101 exhibits an antigenicity similar to the natural Pla I 1 allergen. This allergen induces IgE antibody synthesis that may generate an allergic response in sensitive individuals. Recombinant Pla I 1 protein may be used as the active ingredient in preparations intended for the diagnosis and therapy of allergic diseases induced by Plantago pollens.

Nucleic acid molecule

The nucleic acid molecule of the invention may be a molecule originating from a plant selected from the family Plantaginaceae. The plants of the family Plantaginaceae include Plantaginaceae Arnoglossum S. F. Gray, Plantaginaceae Asterogeum S. F. Gray, Plantaginaceae Bougueria Decne, Plantaginaceae Coronopus Miller, Plantaginaceae Coronopus Reichb., Plantaginaceae Lagopus Fourr., Plantaginaceae Litorella Aschers., Plantaginaceae Littanella Roth, Plantaginaceae Littorella Berg., Plantaginaceae Plantaginella Fourr., Plantaginaceae Plantago L., and Plantaginaceae Plantago Linn., cf. The Plant Names Project (1999), the International Plant Name Index (IPNI), Published on the Internet; http://www.ipni.org (accessed May 21, 2001). Preferably, the nucleic acid molecule of the invention may be a molecule originating from a plant selected from the genus Plantago comprising about 1759 subspecies, cf. the International Plant Name Index (IPNI; www.ipni.org).

The nucleic acid molecule of the invention may be a sequence hybridising to any one of SEQ ID NOS.: 5-7 under stringent conditions, preferably under highly stringent conditions.

Preferably, the said sequence has above about 50%, more preferably above 70%, more preferably above 85%, more preferably above 90 and most preferably above 95% sequence identity with any one of SEQ ID NOS.: 5-7.

Protein or Peptide

As mentioned above the amino acid sequence of the protein or peptide may be modified as compared to SEQ ID NO.: 8 so as to reduce the IgE binding affinity either partly or wholly. Allergens with no IgE binding affinity do not give rise to an antibody-mediated B cell response. However, it is believed that allergens with no IgE binding affinity still may serve as a vaccine, since such allergens are capable of eliciting a T cell response. And even without serological reactivity, it will still be possible to immunise for prophylaxis without the risk of sensitisation.

The recombinant protein or peptide according to the present invention may be produced with a high level of purity. Thus, since recombinant techniques are used to produce the protein/peptide, the resulting product may be produced so as to be totally free of other isoforms of the allergen unlike allergen preparations obtained by extraction of pollen. Moreover, the resulting product may be produced with a purity of above 95% on the basis of total protein. Thus, in a preferred embodiment of the present invention, the protein or peptide has a purity of above 75%, more preferably above 85%, more preferably above 90 and most preferably above 95% on the basis of total protein. In this connection the protein or peptide of the invention includes all forms in which it may be present, including monomeric, dimeric, glycosylated and unglycosylated forms.

The modification of the amino acid sequence may consist in one or more substitutions and/or deletions and/or additions of amino acids.

Preferably, the modified amino acid sequence has a level of identity as compared with any one of SEQ ID NOS.: 5-7 of above 50%, more preferably above about 67%, more preferably above 80%, more preferably above 90% and most preferably above 95%.

Once the amino acid sequence of an allergen is determined, it is possible for a person skilled in the art to determine the position, structure and sequence of the epitopes of the allergen using conventional techniques. The experiments to be carried out in order to determine the position, structure and sequence of the epitopes of the allergen are described in detail in Example 7.

Preferably, the recombinant modified allergen according to invention essentially has the same α-carbon backbone tertiary structure as said naturally occurring allergen.

Specific IgE binding to the modified allergen is preferably reduced by at least 5%, more preferably at more than 25%, more preferably more than 50%, more preferably more than 75% and most preferably more than 90% in comparison to naturally-occurring isoallergens or similar recombinant proteins in an immuno assay with sera from source-specific IgE reactive allergic patients or pools thereof.

Another way of assessing the reduced IgE binding is the capability of the modified allergen to initiate Histamine Release (HR). The release of Histamine can be measured in several Histamine releasing assays. The reduced Histamine release of the modified allergen originates from reduced affinity toward the specific IgE bound to the cell surface as well as their reduced ability to facilitate cross-linking. HR is preferably reduced by 5-100%, more preferably 25-100%, more preferably 50-100% and most preferably 75-100% for the modified allergen of the invention in comparison to the naturally occurring allergens.

A preferred embodiment of the invention is characterised in that one or more of the substitutions is carried out by site-directed mutagenesis.

Another preferred embodiment of the invention is characterised in that one or more of the substitutions is carried out by random mutagenesis.

The modification of the nucleic acid sequence may e.g. be carried out according to the principles set forth in WO 99/47680 and DK patent application PA 200001718.

In a preferred embodiment of the invention, the protein or peptide comprises at least one epitope having the same binding specificity as an epitope on an allergen different from Pla I 1.

Such a hybrid allergen displays the antigenicity of both Pla I 1 and the other allergen in question, and it has the advantage that it may be used to treat or diagnose allergy to both Pla I 1 and the other allergen simultaneously.

Comparing the sequences obtained with other sequences in protein databases, it has been found that among the proteins with which Pla I 1 exhibits the most similarity, Ole e 1 from Olea europaea pollen (identity: about 39%, homology: 68%) is clinically the most important, cf. Example 8. Reference is made to Example 8 for a more detailed account of the sequence similarity between Pla I 1 and its most related allergens.

In a preferred embodiment of the invention, the protein or peptide comprises at least one epitope having the same binding specificity as an epitope on the allergen Ole e 1 from Olea europaea.

As this preferred protein or peptide according to the invention displays the antigenicity of both Pla I 1 and Ole e 1, it has the advantage that it may be used to treat or diagnose allergy to both Pla I 1 and Ole e 1 simultaneously.

It has been shown that Pla I 1 has cross-reactivity with Ole e 1 (30). Thus, the recombinant Pla I 1 allergen provided by the present invention has the advantage that it may be used to effect simultaneous treatment and diagnosis of both Pla I 1 and Ole e 1 allergy.

The recombinant modified allergen according to the present invention may be produced using a DNA sequence obtained by DNA shuffling (molecular breeding) of the nucleic acid molecule encoding Pla I 1. DNA shuffling may be carried out according to the procedures disclosed in the article by Punnonen J: “Molecular Breeding of Allergy Vaccines and Antiallergic Cytokines”. Int Arch Allergy Immunol 2000; 121:173-182 as well as the procedures disclosed in the articles mentioned therein, which are all included herein by this reference.

DNA shuffling may be carried out between the nucleic acid molecule encoding Pla I 1 and any other DNA molecule encoding allergens having a potentially relevant antigenicity to produce DNA hybrids encoding allergenic molecules containing epitopes from two or more different allergens, e.g. epitopes from Pla I 1 and Ole e 1.

In another preferred embodiment of the invention, the protein or peptide is a derivative thereof. Such a derivative may have an unchanged or a reduced IgE binding affinity.

Such derivatives include chemically modified proteins and peptides, e.g. proteins and peptides modified by cross-linking using glutaraldehyde. Such chemically modified proteins and peptides may be obtained by standard protein chemistry methods.

Other chemically modified proteins potentially useful for allergy therapy include allergen-DNA conjugates containing CpG motifs as described in (J.

Allergy Clin. Immunol. 2001; 107; 339-350). The constituents can also be given as mixtures.

Pharmaceutical Composition and Method of Treatment

In addition to the active substance, the pharmaceutical composition of the invention may comprise a number of excipients and adjuvants. The excipient used may be any excipients, which is conventionally used in the formulation of proteins and peptides. The adjuvant may be any adjuvant, which is conventionally used in the formulation of allergens.

Preferably, the pharmaceutical composition of the invention is a vaccine. Preparation of vaccines is generally well known in the art. Vaccines are typically prepared as injectables either as liquid solutions or suspensions. Such vaccine may also be emulsified or formulated so as to enable nasal administration as well as oral, including buccal and sublingual, administration. The immunogenic component in question (the recombinant allergen as defined herein) may suitably be mixed with excipients which are pharmaceutically acceptable and further compatible with the active ingredient. Examples of suitable excipients are water, saline, dextrose, glycerol, ethanol and the like as well as combinations thereof. The vaccine may additionally contain other substances such as wetting agents, emulsifying agents, buffering agents or adjuvants enhancing the effectiveness of the vaccine.

Vaccines are most frequently administered parenterally by subcutaneous or intramuscular injection. In such vaccines the active substance may be absorbed onto a solid support or it may be present in aqueous form. Formulations which are suitable for administration by another route include oral formulations and suppositories. Vaccines for oral administration may suitably be formulated with excipients normally employed for such formulations, e.g. pharmaceutical grades of mannitol, lactose, starch, magnesium stearate, sodium saccharine, cellulose, magnesium carbonate and the like. The composition can be formulated as solutions, suspensions, emulsions, tablets, pills, capsules, microparticles, a liposome preparation, sustained release formulations, aerosols, powders, or granulates. Vaccines according to the present invention may e.g. be formulated according to the principles described in WO 00/45847 and DK patent application PA 200001194.

The vaccines are administered in a way so as to be compatible with the dosage formulation and in such amount as will be therapeutically effective and immunogenic. The quantity of active component contained within the vaccine depends on the subject to be treated, i.a. the capability of the subject's immune system to respond to the treatment, the route of administration and the age and weight of the subject. Suitable dosage ranges can vary within the range from about 0.0001 μg to 1000 μg.

As mentioned above, an increased effect may be obtained by adding adjuvants to the formulation. Examples of such adjuvants are aluminum hydroxide and phosphate (alum) or calcium phosphate as a 0.05 to 0.1 percent solution in phosphate buffered saline, synthetic polymers of sugars or polylactid glycolid (PLG) used as 0.25 percent solution. Mixture with bacterial cells such as C. parvum, endotoxins or lipopolysaccharide components of gram-negative bacteria, emulsion in physiologically acceptable oil vehicles such as mannide monoaleate (Aracel A) or emulsion with 20 percent solution of a perfluorocarbon (e.g. Fluosol-DA) used as a block substitute may also be employed. Oil emulsions, such as MF-59 may also be used. Other adjuvants such as Freund's complete and incomplete adjuvants as well as saponins, such as QuilA, Qs-21 and ISCOM, and RIBI may also be used.

Most often, multiple administrations of the vaccine will be necessary to ensure an effect. Frequently, the vaccine is administered as an initial administration followed by subsequent inoculations or other administrations. The number of vaccinations will typically be in the range of from 1 to 50, usually not exceeding 35 vaccinations. Vaccination will normally be performed from biweekly to bimonthly for a period of 2 months to 5 years. This is contemplated to give desired level of prophylactic or therapeutic effect.

The recombinant allergen may be used as a pharmaceutical preparation, which is suitable for providing a certain protection against allergic responses during the period of the year where symptoms occur (prophylaxis). In some cases, the treatment will have to be repeated every year to maintain the protective effect. Preparations formulated for nasal, oral and sublingual application are particular suited for this purpose.

Finally, the recombinant allergen of the invention may be provided as a combination with a targeting molecule for delivery to specific cells of the immune system or to mucosal surfaces.

Vector and Host

The expression vector according to the present invention may be any expression vector capable of expressing Pla I 1, including a plasmid or a phage. Preferably, the expression vector according to the invention is a plasmid, e.g. pPIC9, pROEX HT (manufacturer: “Life Technologies”), pGAPZ (manufacturer: “Invitrogen”) and pSFVI (manufacturer: “Life Technologies”).

The host according to the present invention may be any host capable of hosting the vector used, including bacteria cells, mammalian cells and yeast cells. In a preferred embodiment of the invention, the host according to the invention is the yeast Pichia Pastoris or the bacteria E. coli.

Method of Diagnosis

The in vitro diagnostic or prognostic method of the invention may be any immunoassay capable of measuring the level of IgE specific to an allergenic 1 o substance.

Preferably, the in vitro assay is selected among the assays described in WO 94/11734, WO 99/67642 and WO 00/37941, which are incorporated herein by this reference.

The in vivo diagnostic or prognostic method of the invention may be any test capable of assessing the sensitivity of a subject to an allergenic substance, such as a cutaneous test, e.g. a skin prick test and intradermal test, and bronchial provocation etc.

The method of predicting the effect of allergy vaccination may be any known method capable thereof, such as the method described in WO 99/67642.

Definitions

In connection with the present invention the expression “epitope” means an antibody-binding structure in the form of either of a fragment of the primary amino acid sequence at least 5 amino acids or of a surface-exposed region of the mature folded protein (three-dimensional, tertiary structure) composed of at least five amino acids. The term “epitope” includes both B-cell and T-cell epitopes. The said antibody may be any immunoglobulin, including immunoglobulins belonging to the classes IgA, IgD, IgE, IgG and IgM.

The expression “fragment of the sequence SEQ ID NOS.: 5, 6 or 7” means a fragment comprising at least 15 base pairs.

The expression “the nucleic acid molecule has a sequence encoding the amino acid sequence . . . ” means any nucleic acid molecule sequence encoding the amino acid sequence specified.

The expression stringent conditions mean the following conditions: a salt concentration of 0.15 M-0.9 M NaCl and a temperature of from 20° C. to 55° C.

The expression highly stringent conditions mean the following conditions: a salt concentration of 0.02 M-0.15 M NaCl and a temperature of from 50° C. to 70° C.

The expression “degeneration” means one or more substitutions of the nucleotides in the nucleic acid molecule sequence, which do not change the sequence of amino acids encoded by the nucleic acid molecule sequence.

The expression “the sequence of SEQ ID NOS.: 5, 6 or 7” means any of the three sequence variants shown in SEQ ID NOS.: 5-7. Likewise, the expression “the sequence of SEQ ID NO.: 8” means any of the three sequence variants shown in SEQ ID NO.: 8.

The expression “recombinant protein or peptide” includes synthetic proteins/peptides, i.e. molecules prepared by chemical synthesis, as well as proteins/peptides prepared using recombinant techniques.

The expression “a reduced IgE binding affinity” means that the IgE binding affinity is reduced either partly or wholly.

### EXAMPLES

A more complete understanding of the invention can be obtained by reference to the following specific Examples. These Examples are described solely for purposes of illustration and are not intended to limit the scope of the invention. Although specific terms have been employed herein, such s terms are intended in a descriptive sense and not for purposes of limitations.

Unless specifically indicated, recombinant DNA techniques are performed according to standard procedures as described in (24).

### Example 1

This example describes the isolation of RNA from P. lanceolata pollen, first-strand cDNA synthesis and amplification of a 3′-fragment of Pla I 1-specific cDNA.

Total RNA was extracted from P. lanceolata pollen by extended sonication of pollen grains in a denaturing solution, followed by phenol-chloroform extraction of the suspension. Pollen (0.6 g) was suspended in 7 ml of denaturing solution consisting in 4 M guanidinium thiocyanate, 25 mM sodium citrate pH 7.0, 0.5% (w/v) lauryl sarcosinate and 100 mM β-mercaptoethanol (25), and the suspension was sonicated for 30 min, duty cycle 50% ultrasonic exposure, at setting 9, in a Vibracell sonifier model VC300. 2M sodium acetate pH 4.0 (0.7 ml), water-saturated phenol (7 ml) and chloroform: isoamyl alcohol (49:1 (v/v), 1.4 ml) was added to the sonicated suspension, and it was incubated for 15 min at 4° C. with occasional shaking. Phases were separated by centrifugation at 10,000 g for 20 min at 4° C. The aqueous phase was transferred to a clean tube and the RNA was precipitated by adding the same volume of isopropanol at −20° C. and incubated for 1 h at −20° C. The pellet was dissolved in 0.3 ml of denaturing solution and precipitated again with isopropanol as before. The pellet was washed with 75% ethanol and dried. Finally, the RNA pellet was dissolved in DEPC-treated water and stored frozen in aliquots at −70° C. until use.

First strand cDNA was synthesized from 10 μg of RNA using Superscript reverse transcriptase and the AP primer, both provided with the 3′-RACE system kit (Gibco-BRL), following the manufacturer's instructions. The AP primer consists of 17dT residues extended in the 5′end with restriction sites appropriate for directed cloning (5′GGCCACGCGTCGACTAGTACTTTTTTTTTTTTTTTTT3′) (SEQ ID NO.: 9).

After RNAaseH treatment, Pla I 1-specific cDNA (3′-fragment) was amplified from 2 μl of first-strand cDNA synthesis reaction mixture using a degenerate oligonucleotide (Pla1, Table 1), deduced from the N-terminal amino acid sequence of Pla I 1 at positions 5-10 (23), as the sense primer, and UAP (5′CUACUACUACUAGGCCACGCGTCGACTAGTAC3′) (SEQ ID NO.: 10), supplied with the 3′-RACE System kit, as the antisense primer. In general, the DNA amplifications performed throughout this work were carried out by the polymerase chain reaction (PCR) using the primers at a concentration of 10 μM and 2.5 units of the enzyme Taq DNa polymerase (Gibco-BRL) in a final reaction mixture volume of 50 μl. Conditions for DNA amplification were those recommended by the manufacturer for the enzyme, adjusting the annealing temperature as a function of the specific primer used in each reaction. For this particular reaction the annealing temperature was adjusted to 51° C. A Gene ATAQ (Pharmacia) programmable thermal controller was used in PCR amplications.

The PCR product was analyzed by electrophoresis in 1.2% agarose/TAE gel, showing a size of approximately 700 bp by comparison with molecular weight markers (100 bp ladder, MBI Fermentas), which is in accordance with the expected size estimated from the molecular weight of the protein.

TABLE 1
Sequence of primers designed for cloning,
sequencing and expression of Pla l 1.

| Primer | 5′ [symbol] 3′ Sequence | Use |
|---|---|---|
| Pla 1 | CAYCCNGCNAARTTYCAYGT | 3′ RACE |
| | (SEQ ID NO.: 11) | |
| Pla 2 | CGGAATTTCACTACAATCGGGCCTGCCGC | 5′ RACE |
| | (SEQ ID NO.: 12) | |
| Pla 3 | CCAATTGCACTTGTGCCCCTGCCATGCGTTCG | 5′ RACE |
| | C | |
| | (SEQ ID NO.: 13) | |
| Pla 4 | ACACAAACATCTCATCCCGC | 3′ RACE |
| | (SEQ ID NO.: 14) | |
| Pla 5 | CTCGAGAAAAGAGAGACACAAACATCTCATCC | Expression |
| | CGC | |
| | (SEQ ID NO.: 15) | |
| Pla 6 | GGGAATTCTTAACACCCAGGGGC | Expression |
| | (SEQ ID NO.: 16) | |

N means that any of A, T, C, or G can be at that position
Y means that any of C or T can be in that position
R means that any of A or G can be in that position

### Example 2

This example describes the cloning and sequencing of a 3′-fragment of Pla I 1-cDNA.

The 3′-fragment of Pla I 1-cDNA amplified by 3′-RACE PCR was purified from agarose gels and used for cloning into pGEM T Easy vector (Promega) with compatible T nucleotide overhanging end [26]. This plasmid carries a gene for ampicillin resistance to enable the selection of transformants and several restriction sites placed to both sides of the cloning site. The ligation reaction was carried out by incubation for 16 h at 4° C. in the presence of T4 DNA ligase (Promega). The ligation products were transformed in the E coli strain DH5α. Plasmidic DNA was prepared from the recombinant transformants using Wizard Plus Minipreps (Promega). To verify that the selected transformants had the insert, a sample from plasmidic DNA was digested with EcoRI (Boehringer) and analyzed by agarose gel electrophoresis. The complete nucleotide sequences of both strands from several clones were determined by the di-deoxynucleotide chain-terminating method [27]. DNA sequencing was performed employing ABI PRISM Dye Terminator system and an ABI 377 automated sequencer (Applied Biosystem).

The sequence of the cDNA fragment coding Pla I 1 had 3′-unstranslated sequences followed by a poly A tail. Some differences between the distinct clones were observed both in the coding and the non-coding region. These differences will be disclosed and discussed in larger extension when referring to the sequences of clones encoding the complete Pla I 1 allergen (Example 4). The sequences obtained allowed us to design specific primers from sequence stretches with no polymorphic positions. These primers were used for 5′-RACE in order to unambiguously elucidate the nucleotide sequence corresponding to the N-terminal end of the protein and the signal peptide (Pla 2 and Pla 3, Table 1), as described in the following example.

### Example 3

This example describes the synthesis, amplification, cloning and sequencing of a 5′-fragment of Pla I 1-cDNA comprising the leader peptide and the N-terminal end of the protein.

First strand cDNA synthesis and amplification of a 5′-fragment of Pla 1-cDNA was achieved using the 5′RACE system kit (Gibco-BRL), and the specific primers derived from the sequence of a 3′-fragment of Pla I 1-cDNA obtained as described in the Example 2 (Pla 2 and Pla 3, Table 1). Briefly, first strand cDNA was synthesized from 10 μg of RNA using a gene-specific antisense primer (Pla 2, Table 1) and Superscript reverse transcriptase (Gibco-BRL). After first strand cDNA synthesis, the original mRNA template was removed by treatment with RNAase Mix (mixture of RNAase H and RNAase T1, Gibco-BRL). Unincorporated dNTPs, Pla 2, and proteins were separated from cDNA using a GlassMax Spin Cartridge provided with the kit. A homopolymeric tail was then added to the 3′-end of the cDNA using Terminal deoxynucleotidyl transferase and dCTP. The tailing reaction was carried out by incubation on ice for 1 h. Since this tailing reaction was performed in a PCR-compatible buffer, an aliquot portion of the reaction was used directly for amplification by PCR without intermediate purification steps. dC-cDNA was amplified using Taq DNA polymerase, a nested, Pla I 1-specific primer (Pla 3, Table 1), and a deoxyinosine-containing anchor primer (AP2, 5′GGCCACGCGTCGACTAGTACGGGIIGGGIIGGGIIG3′) (SEQ ID NO.: 17) provided with the system. For this reaction the annealing temperature was set at 65° C. The PCR product was analyzed by electrophoresis in 1.2% agarose/TAE gel, showing a size of approximately 300 bp by comparison with molecular weight markers, which was in accordance with the expected size. The 5′-fragment of Pla I 1-cDNA amplified by PCR was purified from agarose gels and used for cloning into pGEM T Easy vector (Promega) and sequenced as described in the Example 2. The complete nucleotide sequences of both strands from three clones were determined by the di-deoxynucleotide chain-terminating method. These sequences are indicated in SEQ ID NOS.: 1-3. The nucleotide sequence encoding the leader peptide has the same length (75 bases corresponding to 25 amino acid residues) in all the clones sequenced. Only one base substitution in the leader peptide sequence stretch that implies an amino acid change (SEQ ID NO.: 4) was observed in one clone (clone 14). Regarding the sequence stretch corresponding to the mature protein, no variability was found in the first 86 bases. In position 87 a nucleotide change was observed in clone 14. This change will be deeply discussed in Example 4. A non-degenerate 20-nucleotide length primer (Pla 4, Table 1) was designed from the unambiguous sequence established for the N-terminal end of the mature protein. This primer was used for 3′ extension in order to obtain full-length clones encoding the mature protein that would be used for sequencing as well as for Pla I I expression, as described in Examples 4 and 5.

### Example 4

This example describes the cloning and sequence of Pla I 1-specific cDNA starting from the N-terminal end of the mature protein.

First strand cDNA synthesis from pollen RNA and 3′RACE amplification of Pla I 1-specific cDNA was carried out as described in Example 1, except for the fact that the primer used was Pla 4 (Table 1), corresponding to the positions 1-7 of the amino acid sequence of the mature Pla I 1 allergen, and the annealing temperature for amplifications was set at 55° C. PCR products were purified from agarose gels and cloning into pGEM T Easy vector and sequencing were carried out as described in Example 2. The complete nucleotide sequences of both strands from several clones were determined by the di-deoxynucleotide chain-terminating method. The length of the nucleotide sequences ranged between 674 and 704 bp. These differences are caused by the distinct length of the poly-(A) tract in each clone and some divergences in the 3′-untranslated region. On the basis of nucleotide sequence analysis, these cDNA clones were classified into three groups, each one encoding for an isoallergenic variant of Pla I 1. In SEQ ID NOS.: 5-7, the nucleotide sequence for one clone representative of each group is given. The sequences of these clones showed that all of them coded for a 131 amino acid residues mature protein. In the coding region, sequence polymorphism was observed at four positions (87, 173, 244, 297). The polymorphism at position 87 had also been observed in clones obtained after 5′RACE amplification (Example 3). The nucleotide substitution at that position as well as the change in position 297 do not lead to an amino acid change in the deduced amino acid sequence. However, polymorphisms at positions 173 and 244 imply an amino acid change at positions 58 and 82, respectively, of the amino acid sequence. The comparison of the deduced amino acid sequence for the mature protein encoded by the representative clones of the three groups of clones sequenced is depicted in SEQ ID NO.: 8. Clone 1.2 codes for an amino acid sequence with a glutamic acid at position 58 and a serine at position 82. Clone 1.6 codes for a glycine at position 58 and serine at position 82, and clone 1.4 codes for glycine at both positions. According to the rules of the IUIS for the nomenclature of allergens, the proteins encoded by these clones should be considered as variants. Therefore, they have been named as follows: Pla I 1. 0101 is the allergen encoded by the group of clones represented by Pla I 1.2; Pla I 1. 0102 is the allergen encoded by the group of clones represented by Pla I 1.6; and Pla I 1. 0103 is the allergen encoded by the group of clones represented by Pla I 1.4. All of the variants displayed six cysteine residues in the sequence and a potential N-glycosylation site at position 107. The molecular weight values estimated for the polypeptide backbone of the mature Pla I 1 allergen are 14521, 14463 and 14433 for variants 0101, 0102, and 0103, respectively.

Comparison of the hydrophilicity profiles deduced from the amino acid sequence of the Pla I 1 variants showed that the changes in the amino acid sequence do not imply any significant change in their surfaces properties, and hence that they probably do not modify the antigenic features of the protein.

### Example 5

This example describes the expression of the recombinant Pla I 1 allergen, variant 0101 in the yeast Pichia pastoris.

P. pastoris is a methylotrophic yeast that can use methanol as carbon source whenever there is not any other available. Two genes encoding proteins with alcohol oxidase activity, AOX1 and AOX2, are present in P. pastoris genome. When methanol is the only carbon source available, AOX1 product represents about 80% of the total protein expressed. Taking advantage of this feature, the P. pastoris expression system basically consists in the substitution of AOX1 gene for the gene of interest. On the other hand, the growth of the yeast strain used, GS115 his4, is dependent on the availability of histidine in the culture medium.

The expression vector pPIC9 (Invitrogen) used for expression in P. pastoris carries the DNA coding for the leader sequence of the Saccharomyces cerevisiae α-mating factor in front of the multiple cloning site where the insert is integrated. This signal peptide is efficiently recognized by the yeast, allowing the secretion of heterologous proteins in high yields. The signal peptide encoding DNA and the multiple cloning site are allocated between the 5′ end and the 3′ end of the AOX1 locus, to lead the recombination. Moreover, the vector contains a bacterial replication origin, an ampicilline resistance gene (for selection of transformants in bacteria), and the histidol dehydrogenase gen HIS4 to enable cell growth in a histidine-free culture medium, in order to select transformants in yeast.

For expression, the coding region of the Pla I 1 gene was first amplified by PCR (with the commercial 3′-RACE System kit from Gibco-BRL) using first strand cDNA from pollen mRNA as template and two non-degenerate primers, a sense primer CTCGAGAAAAGAGAGACACAACATCTCATCCCGC (Pla 5) (SEQ ID NO.: 15), and an anti-sense primer GGGAATTCTTMCACCCAGGGGC (Pla 6) (SEQ ID NO.: 16), which, respectively, hybridize with the 5′ and 3′ ends of the protein-encoding regions, in-frame with the sequence coding for the preprosequence of the α-mating factor, present in plasmid pPIC9. Pla 5 also includes a Xho I restriction site (underlined) and a codon for glutamic acid that enables the processing of the signal peptide by the yeast. The anti-sense primer contains a stop codon and an EcoRI restriction site (underlined). The annealing temperature for amplifications was set at 65° C.

PCR products were isolated from agarose gels and used directly for ligation into pGEM T Easy vector with compatible T nucleotide overhanging end, as described in Example 2. This construction was used to transform DH5α E. coli cells, and the transformants were selected by ampicilline resistance. The nucleotide sequences of both strands from several clones were determined by the dideoxynucleotide chain-terminating method, confirming the in-frame arrangement of the leader sequence and Pla I 1, as well as the absence of any change from the starting sequence.

Plasmid DNA was isolated and digested with XhoI-EcoRI restriction enzymes. The DNA fragments were subcloned into the same sites of plasmid pPIC9 rendering pPIC9/Pla I 1.0101.

Plasmid pPIC9/Pla I 1.0101 was linearized with Bgl II restriction enzyme, and the purified larger fragment was used to transform GS115 cells by electroporation. Transformed cells were incubated on minimal dextrose plates at 30° C. for 4-6 days until colonies appeared. Screening for gene replacement of the construct by homologous recombination at the AOX1 locus, rendering a (His⁺ Mut^{s}) phenotype, was performed by patching the His⁺ colonies in replica plating on minimal dextrose vs. minimal methanol plates. Those transformants with retarded growth rate were selected for rPla I 1 production.

Selected (His⁺ Mut^{s}) transformed strains were cultured for four days at 30° C. in buffered glycerol complex medium. Cells were then collected by centrifugation and resuspended in one-fifth of the original volume of buffered methanol complex medium for induction of the AOX1 promoter. This culture was maintained for 4 days and supplemented daily with 5 ml of methanol per litre of culture. The culture medium of GS115-induced cells was cleared of yeast cells by centrifugation at 3,000 g at 4° C. The production of rPla I 1 in the supernatant of the culture medium was analysed at different times by SDS/PAGE and an ELISA with monoclonal antibodies specially designed for quantitation of Pla I 1 (29). The highest expression level of recombinant Pla I 1 was reached at day four after induction.

Large-scale production of rPla I 1 was performed under similar conditions using the colonies that rendered the best yields in the small-scale experiments. A yield of 20 mg of the recombinant allergen was obtained per litre of culture medium.

Example 6

This example describes the purification of recombinant Pla I, variant 0101, from culture medium, and the characterization of the purified protein.

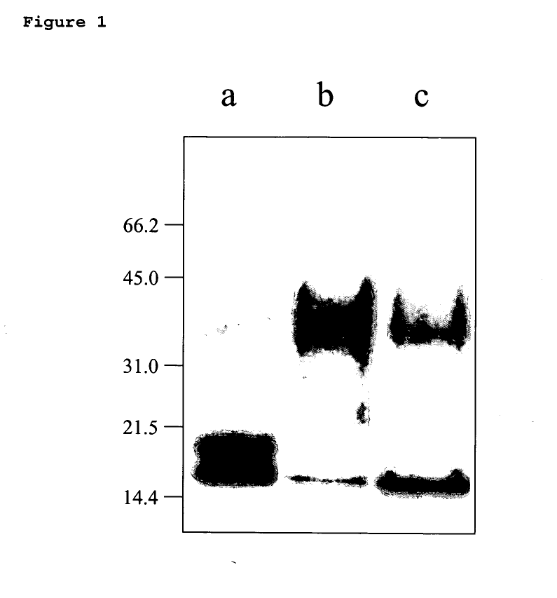

Culture medium obtained as described in Example 5 was dialyzed against water and used as the starting material to purify rPla I 1. Purification was carried out by anionic exchange chromatography using a DEAE-5PW column (Waters Chromatography) with a sodium acetate salt gradient in Tris buffer. The purified recombinant allergen was then dialysed extensively against water. Alternative purification methods, that had been employed for purifying the natural allergen, such as size exclusion chromatography (23) and affinity chromatography with monoclonal antibodies (29), were also applied for the recombinant allergen yielding this in a high degree of purity. The analysis of the purified recombinant allergen Pla I 1 in SDS-PAGE showed an electrophoretic pattern similar to that of the natural Pla I I allergen, with two major bands with apparent molecular weight of 17 and 22 kDa, corresponding to the glycosylated and non-glycosylated monomeric forms of the allergen (FIG. 1), as it was demonstrated by deglycosylation experiments with PNGase F (Boehringer Mannheim). Treatment with this enzyme caused the conversion of the 22 kDa band into the 17 kDa (non-glycosylated) band (FIG. 1). These results were confirmed by MALDI-TOF mass spectrometry. This also demonstrated that a broad diffused band detected in the range of molecular weight 32 to 36 kDa in SDS-PAGE analysis was originated from association of monomer units into a dimer. N-terminal amino acid sequencing and amino acid composition analysis of the purified recombinant allergen confirmed that the protein expressed in P. pastoris was Pla I 1. An additional glutamic acid residue was disclosed at the N-terminus of the protein as a result of the modifications included in the construction of the recombinant DNA for expression in the yeast.

The techniques used to characterize the expressed recombinant allergen also provided information about its degree of purity. Thus, densitometry of Coomassie Blue stained SDS-PAGE gels showed that rPla I 1 was >95% pure. A similar value for purity was deduced after integration of HPLC chromatograms and MALDI-TOF mass spectra. Additionally, only one sequence, with no detectable contaminants, was obtained by Edman degradation of the protein. This indicates that the product in fact has a purity of above 99%.

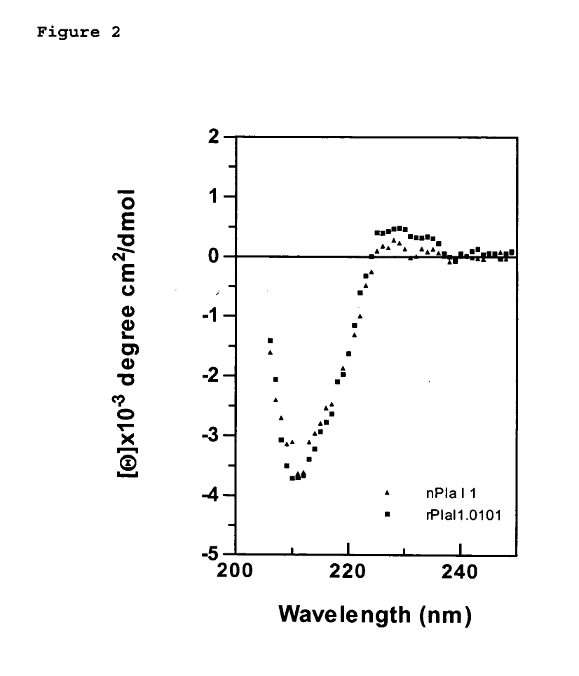

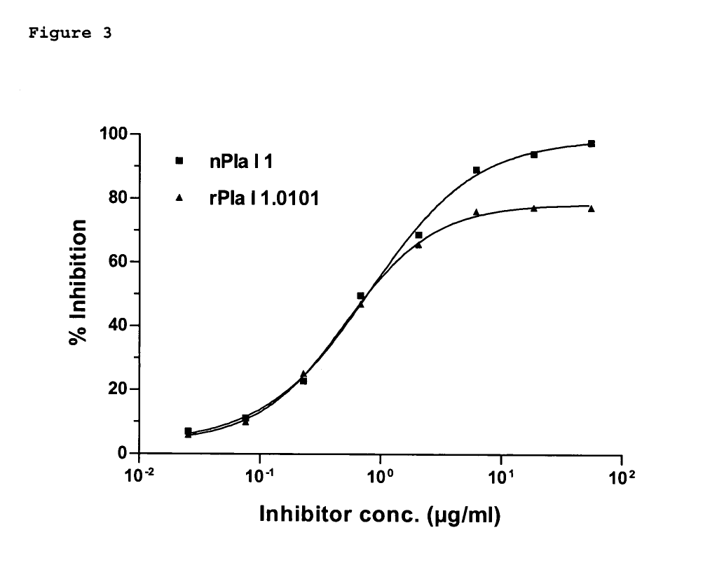

Recombinant Pla I 1 was also immunochemically characterized by using monoclonal antibodies and sera from plantain-allergic patients. The recombinant protein is recognized by the monoclonal antibody 2A10 (29) raised against the natural allergen, thus demonstrating that it bears the same antigenic determinant. This suggests that the folding of the recombinant protein is similar to that of the natural allergen. Results from circular dichroism experiments back up this assumption, as the CD spectra in the far UV of the recombinant allergen was undistinguishable from that of the natural allergen (FIG. 2). On the other hand, rPla I I was tested against a battery of individual sera from plantain-allergic patients, and most of them gave a positive response. Moreover, in an inhibition experiment for the binding of specific IgE from a pool of sera to nPla I 1, the recombinant allergen gave an inhibition curve that was parallel to that of the natural allergen and it could reach up to 80% inhibition of IgE-binding (FIG. 3). All these results demonstrate that most allergenic epitopes present in the natural allergen are conserved in the recombinant allergen, and therefore it could be an adequate tool for diagnosis of plantain-allergic patients.

### Example 7

This example describes the methods for determining the position, structure and sequence of the epitopes of the allergen Pla I 1.

Determination of the position and sequence of sequential epitopes of Pla I 1 is achieved by using overlapping peptides spanning the complete amino acid sequence. The amino acid sequence of these peptides is deduced from the sequence indicated in SEQ ID NO.: 8. These peptides is chemically synthesized or produced as a recombinant peptide by inserting the corresponding nucleotide sequence in an appropriate vector and expressed in a host. B-cell epitopes is identified by detecting those peptides with ability to bind specific antibodies (IgE from serum of allergic patients, monoclonal antibodies, etc.) in immunoassays or other immunochemical techniques. Moreover, the peptides is tested in T-cell proliferation assays in order to detect those that form part of T-cell epitopes.

The availability of the nucleotide sequence of Pla I 1 and the expressed recombinant allergen facilitates the identification of the conformational epitopes on the allergen. Thus, data on tertiary structure is obtained by structural analysis of the recombinant allergen using X-ray crystallography and nuclear magnetic resonance. Alternatively, the amino acid sequence is analyzed using predictive computer algorithms to target potential surface residues that may form part of an epitope. Then, site-directed mutagenesis is used to generate Pla I 1 variants with substitutions of amino acid residues potentially involved in B-cell epitopes. Analysis of antibody binding capacity of these variants allows the establishment of those residues that constitute the epitope.

Those peptides or rPla I 1 variants with reduced IgE-binding ability constitute excellent candidates for a safer and more effective treatment for plantain allergies.

### Example 8

The three variants of Pla I 1 has been compared to known protein sequences using protein sequence databases. The results are given in Table 2

TABLE 2

| | | | | Pla I | | Pla I | | Pla I | |
|---|---|---|---|---|---|---|---|---|---|
| | | | | 1.0101 | | 1.0102 | | 1.0103 | |
| Allergen | Mm | PI | N ° R | % I | % S | % I | % S | % I | % S |
| Putative Ole e 1 | 18384 | 8.12 | 166 | 42.2 | 72.7 | 41.4 | 71.9 | 40.6 | 71.8 |
| (Betula | | | | | | | | | |
| verrucosa) | | | | | | | | | |
| Lig v 1 | 16400 | 5.91 | 145 | 39.5 | 69.0 | 39.5 | 69.0 | 38.8 | 69.0 |
| Ole e 1 | 16330 | 6.18 | 145 | 38.8 | 68.2 | 38.8 | 68.2 | 39.5 | 68.2 |
| Lol p 11 | 14876 | 5.10 | 134 | 28.8 | 50.8 | 28.0 | 50.0 | 49.2 | 27.3 |

Table 2: Molecular characteristics of allergens homologous to Pla I 1. Sequence identity (% I) and similarity (% S) percentages. PI, isolectric point; Mm, molecular mass; N^{o}R, number of residues. Amino acid sequences of the three first allergens are in the Swiss-Prot Data Bank. Accesion numbers are: 049813 for birch putative Ole e 1, 082016 for Lig v 1, and P19963 for Ole e 1. Lol p 11 sequence is in the NCBI Data Bank, and the accession number is A54002.

### REFERENCES

1. Jarvis D, Burney P (1997) Epidemiology of atopy and atopic disease. In Allergy and Allergic Diseases, 1^{st} edn, pp 1208-24. Blackwell Science, Oxford.

2. Bousquet J, Lockey R F, Mailing H-J. WHO Position Paper. Allergen Immunotherapy: therapeutic vaccines for allergic diseases. Allergy 1998; 53 Suppl:1-42.

3. Carreira J, Lombardero M, Ventas P. New developments in in vitro methods. Quantification of clinically relevant allergens in mass units. Arb Paul Ehrlich Inst Bundesant Sera lmpstoffe Frank A M 1994; 87:155-64.

4. Valenta R, Vrtala S, Laffer S, Spitzauer S, Kraft D. Recombinant allergens. Allergy 1998; 53:552-61.

5. Spieksma FThM, Charpin H, Nolard N, Stix E. City spore concentration in the European Economic Community (EEC). Clin Allergy 1980;10:319-29.

6. Negrini A C, Arobba D. Allergenic pollens and pollinosis in Italy: recent advances. Allergy 1992;47:371-9.

7. Peat J K, Woolcock A J. Sensitivity to common allergens: relation to respiratory symptoms and bronchial hyper-responsiveness in children from three different climatic areas of Australia. Clin Exp Allergy 1991;21 :573-81.

8. Lewis W H, Imber W E. Allergy epidemiology in the St. Louis Mo. area. IV. Weeds. Ann Allergy 1975; 35:180-7.

9. Bernton H S. Plantain hay fever and asthma. JAMA 1925;84:944-6.

10. Blumstein G I, Tuft L. Plantain hay fever. Its incidence and importance. JAMA 1937; 108:1500-2.

11. Derrick E. Relative importance of various plants in causation of hay fever and asthma in Australia. Med J Aus 1962;1:972-7.

12. Bryant D H, Burns M W. Skin-prick test reactions to inhalant allergens in asthmatic patients. Med J Aust 1976;1:918-24.

13. Krilis S, Baldo B A, Basten A. Detailed analysis of allergen specific IgE responses in 341 allergic patients. Associations between allergens and between allergen groups and clinical diagnoses. Aust NZJ Med 1985; 15:421-6.

14. D'Amato G, Lobefalo G. Allergenic pollens in the southern Mediterranean area. J Allergy Clin Immunol 1989; 83:116-22.

15. Bousquet J, Cour P, Guerin B, Michel F B. Allergy in the Mediterranean area 1. Pollens counts and pollinosis of Montpellier. Clin Allergy 1984; 14:249-58.

16. Merrett T G, Pantin C F A, Dimond A H, Merrett J. Screening for IgE-mediated allergy. Allergy 1980;35:491-501.

17. Mehta V, Wheeler A W. IgE-mediated sensitization to English Plantain pollen in seasonal respiratory allergy: identification and partial characterization of its allergenic components. Int Arch Allergy Appl Immunol 1991;96:211-7.

18. Subiza J, Jerez M, Jimenez J A, Narganes M J, Cabrera M, Varela S, Subiza E. Clinical aspects of allergic disease. Allergenic pollen and pollinosis in Madrid. J Allergy Clin Immunol 1995;96:15-23.

19. González F J, Herrero B, Candau P. Latitudinal study of allergic pollen in two Spanish cities. J Invest Allergol Clin Immunol 1993;3:304-10.

20. Garcia-González J J, Vega-Chicote J M, Rico P, Moscoso del Prado J, Carmona M J, Miranda A et al. Prevalence of atopy in students from Málaga, Spain. Ann Allergy Asthma Immunol 1998; 80: 237-44.

21. Baldo B A, Chense Q J, Howden M E H, Sharp P J. Allergens from Plantain (Plantago lanceolata). Studies with pollen and plant extracts. Int Archs Allergy Appl Immun 1982;68:295-304.

22. Dreborg S, Sjogren I, Erikson E, Einarsson R. Selection of patients for biological standardization as exemplified by standardization of mugwort, goosefoot and English Plantain pollen allergen extracts/preparations. Allergy 1987;42:485-95.

23. Calabozo B, Barber D, Polo F. Purification and characterization of the main allergen of Plantago lanceolata pollen, Pla I 1. Clin Exp Allergy 2001; 31:322-30.

24. Sambrook J, Fritsch E F, Maniatis T (1989) Molecular Cloning: a Laboratory Manual, 2^{nd} edn. Cold Spring Harbor Laboratory Press, Cold Spring Harbor, N.Y.

25. Chomczynski P, Sacchi N. Single-step method of RNA isolation by acid guanidinium thiocyanate-phenol-chloroform extraction. Analyt Biochem 1987; 162,156-9.

26. Marchuk D, Drumm M, Saulino A, Collins F S. Construction of T-vectors, a rapid and general system for direct cloning of unmodified PCR products. Nucleic Acids Res 1991; 19,1154.

27. Sanger F, Nicklen S, Coulson A R. DNA sequencing with chain-terminating inhibitors. Proc Natl Acad Sci USA 1977; 74, 5463-7.

28. King T P, Hoffman D, Lowenstein H, Marsh D G, et al. Allergen nomenclature. Clin Exp Allergy 1995; 25: 27-37.

29. Calabozo B, Duffort O, Carpizo J A, Barber D, Polo F. “Monoclonal antibodies against the major allergen of Plantago lanceolata pollen, Pla I 1. Affinity chromatography purification of the allergen and development of an ELISA method for Pla I 1 measurement” Allergy 2001; 56:429-35.

30. Calabozo B, Diaz-Perales A., Salcedo G. Barber D., Polo F. “Structural and antigenic similarity between Pla I 1 and Ole e 1, the major allergens of English Plantain and Olive pollens”. Abstract No. 51, Journal of Allergy and Clinical Immunology (2001), Vol.107, No. 2, page S15.

1. A nucleic acid molecule encoding a peptide or protein comprising at least one epitope of the major allergen of Plantago lanceolata, Pla I 1, wherein the nucleic acid molecule a) has the sequence set forth in any one of SEQ ID NOS.: 5-7, b) is a fragment of the sequence set forth in any one of SEQ ID NOS.: 5-7, c) has a sequence encoding the amino acid sequence of SEQ ID NO.: 8 or a fragment thereof, d) has a sequence hybridising to set forth in any one of SEQ ID NOS.: 5-7 under stringent conditions, e) has a sequence derivable by degeneration set forth in any one of SEQ ID NOS.: 5-7, or f) a complementary strand of any of the sequences a)-e).

2. The nucleic acid molecule according to claim 1, wherein nucleic acid molecule originates from a plant selected from the family Plantaginaceae.

3. The nucleic acid molecule according to claim 1, wherein sequence d) is a sequence hybridising to the complement of a nucleic acid selected from SEQ ID NOS.: 5-7 under highly stringent conditions.

4. The nucleic acid molecule according to claim 1, wherein sequence d) has at least about 67% sequence identity with a nuclueic acid selected from SEQ ID NOS.: 5-7.

5. A recombinant protein or peptide comprising at least one epitope of the major allergen of Plantago lanceolata, Pla I 1 having the amino acid sequence corresponding to the nucleic acid sequence of claim 1 disclaiming the amino acid sequence consisting of amino acids 1-16 of SEQ ID NO.: 8 and fragments thereof.

6. The protein or peptide according to claim 5 comprising a modified amino acid sequence as compared to SEQ ID NO.:8 and having a reduced IgE binding affinity.

7. The protein or peptide according to claim 5 comprising at least one epitope having the same IgE binding specificity as an epitope on the allergen Ole e I from Olea europaea.

8. The protein or peptide according to claim 5, wherein the protein or peptide is a derivative thereof.

9. The protein or peptide according to claim 5 for use as a pharmaceutical.

10. An expression vector adapted for transformation of a host, the vector comprising a nucleic acid molecule according to claim 1.

11. The expression vector according to claim 10, wherein the vector is a plasmid.

12. A host cell comprising the expression vector according to claim 10.

13. A method of producing a recombinant peptide or protein comprising at least one epitope of the major allergen of Plantago lanceolata, Pla I 1,the method comprising culturing the host cell of claim 12 under conditions such that said Pla I 1 nucleotide sequence is expressed and said peptide or protein is produced, and isolating said peptide or protein.

14. A pharmaceutical composition comprising as an active substance a recombinant peptide or protein according to claim 5.

15. A method of preventing, alleviating or treating allergic reactions in a subject comprising administering to the subject a recombinant peptide or protein according to claim 5.

16. A method of preventing, alleviating or treating allergic reactions in a subject comprising administering to the subject a pharmaceutical composition according to claim 15.

17. An in vitro method of diagnosing or prognosticating allergy to Pla I 1 allergen in a subject comprising collecting a sample from the subject and determining the level of IgE antibodies to the protein or peptide according to claim 5.

18. An in vivo method of diagnosing or prognosticating allergy to Pla I 1 allergen in a subject comprising subjecting a subject to the protein or peptide according to claim 5 and monitoring the reaction of the subject.

19. A reagent for use in in vitro or in vivo diagnosing or prognosticating allergy to Pla I 1 allergen in a subject, wherein the reagent contains the protein or peptide according to claim 5.

20. A method of predicting the effect of allergy vaccination comprising using the protein or peptide according to claim 5.
